(19) United States
(12) Patent Application Publication
(10) Pub. No.: US 2017/0307597 A1
(43) Pub. Date: Oct. 26, 2017

(54) Platform and Methods for Analyzing Effects of Genetics, Age and Environment in Stimulus-Response Studies Using Induced Pluripotent Stem Cells

(71) Applicant: Coyne IP Holdings, LLC, Smyrna, GA (US)

(72) Inventors: Kevin P. Coyne, Atlanta, GA (US); Shawn T. Coyne, Smyrna, GA (US)

(21) Appl. No.: 15/518,381

(22) PCT Filed: Oct. 15, 2015
(86) PCT No.: PCT/US2015/055725

§ 371 Date: Apr. 11, 2017

Related U.S. Application Data
(60) Provisional application No. 62/064,365, filed on Oct. 15, 2014.

(51) Int. Cl. G01N 33/50

(52) U.S. Cl. CPC G01N 33/5073

(57) ABSTRACT

Platforms and methods for conducting stimulus-response studies on induced pluripotent stem cells, or cells differentiated therefrom, that have been derived from cells collected from donors. Samples are taken at various ages and/or before and after exposure to environmental conditions. Methods for preparing the platforms are also provided. In one embodiment, multiple donors are involved. In another embodiment, induced pluripotent stem cells are differentiated. Optionally, the induced pluripotent stem cells or differentiated cells thereof, are preserved for subsequent analysis.

### FIELD OF THE INVENTION

The present application is directed to methods for determining the effect of genetics, age and environment in stimulus-response studies using induced pluripotent stem cells or cells differentiated therefrom.

### BACKGROUND

The scientific community has long been interested in the underlying causes of biological responses of cells and tissues to various stimuli, such as the administration of pharmaceuticals, and exposure to chemicals, bacteria and viruses. Scientists have recognized that the following factors play key roles in the response: the nature of the stimulus; the genetic profile of the test subject; the age of the test subject; and the unique history of physical exposures, such as diet, disease, chemicals, and environmental insults, that the subject has endured throughout its lifetime. However, scientists have had difficulty sorting out the contributions of the various factors because any available cells or tissues from a test subject, and the associated testing methodologies, inevitably exhibited an idiosyncratic combination of all four factors.

Scientists in certain fields such as pharmaceuticals and environmental toxicology have been particularly interested in determining and understanding the impact of age, and the impact of a history of various types of exposures, on a test subject's reaction to a particular stimulus, because there are numerous examples of such impacts having important effects on human health. For example, studies have shown that smoking subsequently increases the harm of exposures to asbestos, alcohol, arsenic, and nickel, while other studies have shown that smoking exacerbates the impact of later Hepatitis B and HPV infections.

Despite the sporadic demonstrations of exposure history altering a biological response to subsequent stimuli, the scientific community's knowledge about these types of effects is clearly incomplete, both in coverage and in the level of confidence about many aspects of the effects that have been identified.

The primary tool of the scientific community for studying these issues has historically been epidemiological studies, but such studies face several limitations. First, they are limited to studying actual events. Therefore, findings are limited to retrospective events, not prospective ones. Second, any biological “responses” are the results of an amalgam of the precipitating stimulus, but also historical exposures, such as diet, chemical and biological exposure, environmental exposure, etc. The effects of each exposure are difficult to separate from the effects of all other exposures the donor has experienced. There are simply too many potential differences in the nature and degree of exposures, such that each “victim” identified is, in fact, a unique “victim”. Statistical separation based on large numbers of observed “victims” (if, indeed, there are large numbers of “victims” for the stimulus under study) tend to produce only faint correlations. Third, the results are limited to a “pinpoint” observation of one unique permutation of the stimulus itself. By definition, there can be no testing of alternative versions of the stimulus itself, such as increasing or decreasing the extent or intensity of the stimulus.

Another platform that may be considered is the use of primary cells taken from a single donor at two or more points in time. Some scientists might initially be interested in this approach, because it superficially resembles the common practice of taking “before and after” biopsies surrounding medical procedures to help determine if the procedure was beneficial. The resemblance is only superficial, because a single-donor/multi-time period/stimulus-response (SD-MTP-SR) platform is fundamentally different than a set of before-after biopsies or any other before-after tests of a living subject. Before-after constructs assist analysis of the direct effects of a stimulus (e.g., a medical procedure), by asking, for example, whether the procedure itself caused a sought-after change. By definition, when using the before-after technique, the stimulus occurs in vivo between the original and subsequent biopsies. In contrast, an SD-MTP-SR platform assists the analysis of whether an external stimulus would have a different impact on a test subject if it were applied post the time of the first biopsy (but prior to the time of the second biopsy) than it would have if applied only after the time of the second biopsy.

There are significant issues with using primary tissues when seeking to understand the impact of age and exposure history on biological reactions in a stimulus-response study, regardless of whether cadavers or live subjects are used.

Sourcing primary tissues from cadavers usually directly defeats the purpose of the exercise—i.e., to obtain tissues from two different points in the same test subject's history, wherein exposures have changed in between the two samplings. Further, cadaver sourcing usually occurs under circumstances where obtaining a full inventory of the test subject's history of exposures is impossible.

Sourcing suitable primary tissues from live subjects is also fraught with issues. First, many primary tissues are not suitable for biopsies (e.g., eyes, teeth, brain), while others (e.g., heart muscle, liver cells) are not accessible without utilizing severely invasive procedures (and, of course, the comparisons sought here require a minimum of two such invasions). In addition, quantities of tissues obtainable from live subjects are usually small, limiting usefulness. Next, primary cells are often poor candidates for cryopreservation, requiring the practitioner to apply the stimulus to the first set of cells immediately, then to apply the identical stimulus to the second set of cells only much later, thereby potentially reducing the consistency of application. Finally, by definition, the only cell types available are the cell type actually collected. Therefore, there can be no analysis of “side effects” of the stimulus on other, uncollected, types of cells, and it would be difficult to find donors who both match the history of exposures the practitioner wishes to study and also would be willing to participate in the taking of two or more rounds of biopsies of multiple tissues.

Therefore, what is needed is a reliable and accurate method for studying the impact and interactions of genetics, age and/or exposures at different points in time on an individual or population's reaction to a particular stimulus.

### BRIEF SUMMARY OF THE INVENTION

Platforms and methods are provided herein for conducting stimulus-response studies on iPSCs, or cells differentiated from iPSCs, that have been derived from cells collected from donors. Samples are taken at various ages and/or before and after exposure to environmental conditions. Identical-protocol stimulus-response studies are then conducted on the two (or more) sets of cells, and the results contrasted. Methods for preparing the platforms are also provided.

In some embodiments, multiple donors are involved. In some embodiments, iPSCs are differentiated. In some embodiments, the iPSC or differentiated cells thereof, are preserved for subsequent analysis.

It is well known, through epidemiological studies, that increased age and/or differences in exposures to such factors as diet, pharmaceuticals, and the environment can temporarily or permanently alter an organism's biological responses to subsequent stimuli, such as exposures to chemical or biological agents. However, to date there has been no broadly applicable platform for testing such effects on an experimental basis.

Such a broadly applicable platform can be created by obtaining tissue samples from the same individual donor at different points in time (and therefore, by definition, at either different ages, and/or wherein the later tissue sample embeds additional histories of exposures) and converting, reprogramming or inducing, cells from these tissue samples to become induced pluripotent stem cells (iPSCs). When the various sets of the donor's iPSCs are converted to a common functional cell type of interest, and subsequently exposed to an identical stimulus, any differences in biological responses between the earlier and later sets of cells can provide direct experimental evidence of the impact of age and exposure history on biological responses of the functional cells to a given stimulus. This can enable practitioners to create a profile of the impact of age and environment across multiple stimuli in the case of the individual donor. Repeating the process of taking samples at additional, even later, points in a particular donor's life can elucidate the progression of age and exposure effects on responses to particular stimuli.

The single-donor/multi-time-period/stimulus-response (SD-MTP-SR) platform can also be constructed for multiple individual donors for use in a multi-donor study. In such a case, practitioners can develop the experimental data to better isolate the impacts of genetics, age, and certain exposures on biological responses to a given stimulus than has been possible before.

SD-MTP-SR platforms can be created under a variety of specifications, each of which provides different opportunities for creating useful comparisons. For example, practitioners can obtain the first set of cells in a SD-MTP-SR platform at or near the time of birth, in order to create a baseline set of cells derived from cells that have been subjected to neither age nor exposure effects. This can facilitate understanding of the “entirety of age and exposure effects” through comparisons of the reactions of the cells derived from cells collected at birth to the reactions of other sets of cells derived from cells collected at later points in the donor's life. Alternately, practitioners can create a baseline set of cells derived from cells collected immediately before an important exposure (such as the initiation of chemotherapy or radiation treatments for a cancer patient) in order to make comparisons to cells derived from cells collected immediately after the exposure, to attempt to isolate the effects of that particular exposure, and so on.

A system is provided herein containing one or more single-donor/multi-time-period/stimulus-response (SD-MTP-SR) platforms, wherein an individual SD-MTP-SR platform comprises: (a) two or more sets of iPSCs, or cells or tissues derived from those iPSCs, wherein each set has been derived from a set of cells of any type collected from a single donor at a specified time that is different from the time of collection of cells from the same donor in other sets; and (b) a description of any length or format of the time difference between the collections, and/or at least one dietary, chemical, biological or environmental factor of interest to which the donor was exposed during the time between the collection of the first set of cells and the collection of the later set of cells.

When two sets of iPSCs collected at different points in time are converted to a common functional cell type of interest, and exposed to an identical stimulus, the differences in biological responses among cells in four conditions (Pre-Exposure and Pre-Stimulus, Pre-Exposure and Post-Stimulus, Post-Exposure and Pre-Stimulus, and finally Post-Exposure and Post-Stimulus) can provide direct experimental evidence of the impact of age and exposure history on responses to a given stimulus. Repeated use of the platform across multiple experiments can enable practitioners to create a profile of the impact of age and environment across multiple stimuli in the case of the individual. Further, repeating the construction of a single-donor/multi-time-period platform at additional points in a particular test subject's life can elucidate the progression of age and exposure effects on responses to particular stimuli.

As noted above, also provided is an SD-MTP-SR platform constructed using multiple individuals for use in a multi-donor study. In such a case, practitioners can develop the experimental data to better isolate the average (i.e. mean, median or any other measure of centrality), range and/or distribution among the sample of the impacts of genetics, age, and certain exposures on biological responses to a given stimulus than has been possible before.

### BRIEF DESCRIPTION OF THE DRAWINGS

FIG. 1 is a schematic representation of a stimulus-response study showing the response values and temporal relationships of the pre-stimulus condition (pre-S), application of stimulus, and the post-stimulus condition (post-S). Numerical values indicate observational data points.

FIG. 2 is a schematic representation of a stimulus-response study showing the end-point response values and temporal relationships of the pre-stimulus condition (pre-S), application of stimulus, the post-stimulus condition (post-S), the pre-exposure condition (pre-E), exposure experienced, and the post-exposure period (post-E) at various time points. Numerical values indicate observational data points.

FIG. 3 is a schematic representation of a (different) stimulus-response study showing the end-point response values and temporal relationships of the pre-stimulus condition (pre-S), application of stimulus, the post-stimulus condition (post-S), the pre-exposure condition (pre-E), exposure experienced, and the post-exposure period (post-E) at various time points. Numerical values indicate observational data points.

FIG. 4 is a schematic representation of (yet another) stimulus-response study showing the end-point response values and temporal relationships of the pre-stimulus condition (pre-S), application of stimulus, the post-stimulus condition (post-S), the pre-exposure condition (pre-E), exposure experienced, and the post-exposure condition (post-E) at various time points for multiple stimuli. Numerical values indicate observational data points.

FIG. 5 is a schematic representation of a stimulus-response study showing the end-point response values and temporal relationships of the pre-stimulus condition (pre-S), application of stimulus, the post-stimulus condition (post-S), the pre-exposure condition (pre-E), exposure experienced, and the post-exposure condition (post-E) at various time points for multiple test subjects. Numerical values indicate observational data points.

### DETAILED DESCRIPTION OF THE INVENTION

Platforms and methods for conducting single-donor/multi-time period/stimulus-response studies are provided herein in which induced pluripotent cells (iPSCs), or cells derived therefrom, are employed. The iPSCs used in the method have been produced from cells, obtained from one or more pre-selected donor(s) from two or more time periods and/or before and after exposure to a predetermined substance or environment. In some embodiments, multiple donors are involved. In some embodiments, the iPSCs are differentiated. Optionally, the cells, or their progeny or any derivative cells, are preserved, such as by cryopreservation, and subsequently thawed at any appropriate point in the process. Methods for preparing the platforms are also provided.

Systems including one or more single-donor/multi-time period/stimulus-response (SD-MTP-SR) platforms are described herein, wherein an individual SD-MTP-SR platform contains (a) two or more sets of iPSCs (or cells or tissues derived from those iPSCs) wherein each set has been derived from a set of cells of any type collected from a single donor at a specified time that is different from the time of collection of cells in other sets; and (b) a description of any length or format of the time difference between the collections, and/or at least one dietary, chemical, biological or environmental factor of interest to which the donor was exposed during the time between the collection of the first set of cells and the collection of the second (and subsequent) sets of cells.

The method utilizes a single-donor/multi-time period/stimulus-response (SD-MTP-SR) platform wherein the following actions are taken in any appropriate order: (a) a population of cells is collected from a live mammalian donor; (b) the population is converted to iPSCs; (c) optionally, the iPSCs are differentiated into one or more functional cell types; (d) optionally, the collected cells, or the iPSCs, or the functional cells, or any of the three, are cryopreserved and later thawed at any point in the process; (e) one or more populations of cells are collected from the same donor at a later point, or points, in time; (f) the population is converted to iPSCs; (g) optionally, the iPSCs are differentiated into the same functional cell types as were the first population; (h) optionally, the later population(s) of collected cells, or the iPSCs, or the functional cells, or any of the three, are cryopreserved and later thawed; and (i) a description of any length or format of the time difference between the collections, and/or at least one dietary, chemical, biological or environmental factor of interest to which the donor was exposed during the time between the collection of the first set of cells and the collection of the second (and subsequent) sets of cells is obtained.

Definitions

The term “exposome”, as used herein, means the cumulative measure of environmental influences of biological responses throughout the lifespan of a human or other mammal, including but not limited to exposures from behavior, diet, disease, bacteria, viruses, pharmaceuticals, chemicals, and the environment, as well as any previous endogenous responses to these external stimuli which permanently affect subsequent biological responses. Thus, this definition includes not only the direct impact of external challenges, but also the indirect effects of such challenges, such as epigenetic changes, scarring, DNA mutation or larger chromosomal damage.

The term “null-exposome conditions”, as used herein, means conditions wherein a cell, set of cells, or tissues constructed entirely or in part from those cells, in which the experimental response of those cells is unaffected (within the acceptable tolerances prescribed for that experiment) by either age-related effects of the cells themselves, or by exposure of the cells to the exposome. In situations where multiple test subjects are involved, null-exposome conditions include cells that have had some exposure to the exposome, provided that: (1) the exposure is judged be likely to have minimal impact on responses in the experiment or study under investigation; (2) steps have been taken to minimize any differences in exposures among test subjects; and (3) any remaining differences are explicitly judged to fall within acceptable tolerances prescribed for that experiment or study.

The terms “single-donor/multi-time period/stimulus-response platform” or “SD-MTR-SR platform”, as used herein, means a platform consisting of separate sets of cells from the same donor meeting certain specifications wherein the purpose of creating the sets is to conduct stimulus-response experiments, wherein the response may be a function of both the stimulus and exposure to the exposome during the period or periods of time between the collection of the source cells for the various sets from any single donor in the platform.

The terms “stimulus or stimuli”, as used herein, means any external physical material or force that is applied alone, in plurality, or in any combination, and that may cause a reaction in the behavior or structure of the cells or tissues under investigation. Examples of stimuli include, but are not limited to, chemicals, including pharmaceutical compounds and industrial chemicals; biological agents such as bacteria, viruses, molds, mycoplasma, allergens or other pathogens; light; heat; sound; atmospheric pressure; electrical impulses; radiation; physical trauma; and any form of physical stress, including that resulting from externally induced physical activity of the cells themselves.

Method Development

The platform and methods provided herein are useful for studying the biological response of cells, or tissues made from those cells, of an individual test subject, or more than one test subject, to one or more stimuli, when those responses might be affected by age, genetics, or exposures to the Exposome experienced by the test subject(s).

To construct the test platform, the practitioner must develop specifications for a number of attributes of the donors of the cells, the cells themselves, information on the donors' exposures, etc., while also ensuring that he/she can actually obtain the cell samples that meet the specifications. Specifications include:

(i) The number of donors required, due to the purpose of the experiment. For example, the practitioner will consider whether the experiment is intended to measure the responses of one particular individual, or to be a representative sample of a larger population, or to explore the diversity of responses within a population.

(ii) The criteria for inclusion in the donor pool. For example, the practitioner will determine the age requirements, genetic requirements, and exposures to the Exposome that are required/prohibited prior to the initial sampling of cells, and the time and/or exposures that are required/prohibited between the collection of the first cells and the collection of the second and any subsequent cells.

(iii) The cell type(s) necessary for use in the eventual stimulus-response experiment. In one embodiment, the required cell type(s) are created or formed from iPSCs that are transfected from the test cells or sample collected from the donor(s).

(iv) The information on the ages, genetics and exposures that will be accumulated for each donor and sample collection.

(v) The protocols for the actual cell collection, transfection to iPSCs, cryopreservation and storage, and differentiation of cells to the types specified for the stimulus-response experiments, as well as the tests to be applied at each stage to verify that the progression through the stages has resulted in cells that meet the required specifications.

The actual execution of these steps then proceeds using scientific techniques known to those skilled in the art.

The platform and methods provided herein provide several benefits: (1) use of iPSC conversion; (2) the collection of information about the exposures of the donors between the time of initial cell collection and the time of subsequent cell collections; (3) new insights into the effects of exposures on stimulus-response experiments, without being bound by any one mathematical approach to the analysis; and (4) the ability to utilize numerous embodiments. Each of these aspects is discussed in more detail below.

1. iPSC Conversion

The method provided herein may include the step of converting any cells collected from a donor (both initially and subsequent to the exposures of interest) into iPSCs, and then—if appropriate to the stimulus-response experiment of interest—into functional cells. This step is advantageous for at least three reasons.

First, the Pre-Exposure and Post-Exposure cells actually subjected to the stimulus should be of the identical cell type if the practitioner is to attribute differences in response to the intervening age and exposures, rather than differences in the underlying cell type. Practitioners may not always be able to obtain the same cell type at both collection points. For example, the initial cells may have been taken during surgery, and the act of performing a subsequent surgery on the donor simply for the purpose of collecting the same cells would be problematic. The conversion to iPSCs ensures that the initial and subsequent sets of test cells can be as close to identical as necessary to establish comparability.

Second, the number of cells required in the stimulus-response experiments may exceed the number of primary cells that can be obtained, particularly if the practitioner intends to repeat the experiment (e.g., with different stimuli). The iPSC conversions enable the practitioner to have available as many cells as needed.

Third, various stimuli do not cause biological responses in every cell type. By converting the primary cells to iPSCs, the practitioner can then convert the iPSCs into specific cell types that will be responsive in a particular stimulus-response experiment of interest.

2. Collection of Information About the Exposures of the Donors Between the Time of Initial Cell Collection and the time of Subsequent Cell Collections.

The passage of time between the collection of the initial set of cells and the collection of subsequent sets inevitably results in the potential for a variety of exposures, including both those that are the subject of study and those that are not the subject of study but might have effects on the results of the stimulus-response experiment. Therefore, the practitioner may exercise diligence in the recognition, measurement and documentation of the elements of the Exposome that are of direct interest in the study, but also of any elements of the Exposome that, while not of direct interest, carry any significant probability of also affecting results.

3. The Methods Enable New Insights into the Effects of Exposures on Stimulus-Response Experiments, but are not Bound by any One Mathematical Approach to the Analysis

In part, the value of the method derives from the additional data that can inform any subsequent analysis of a stimulus-response experiment versus the data that would have been available without the pairing of Pre-Exposure and Post-Exposure observations.

To illustrate: Consider an experiment in which a practitioner intends to study the impact of a particular exposure (referred to here as E), on the biological response (R) of an individual test subject to a particular stimulus (S). Assume the practitioner believes all effects are strictly additive.

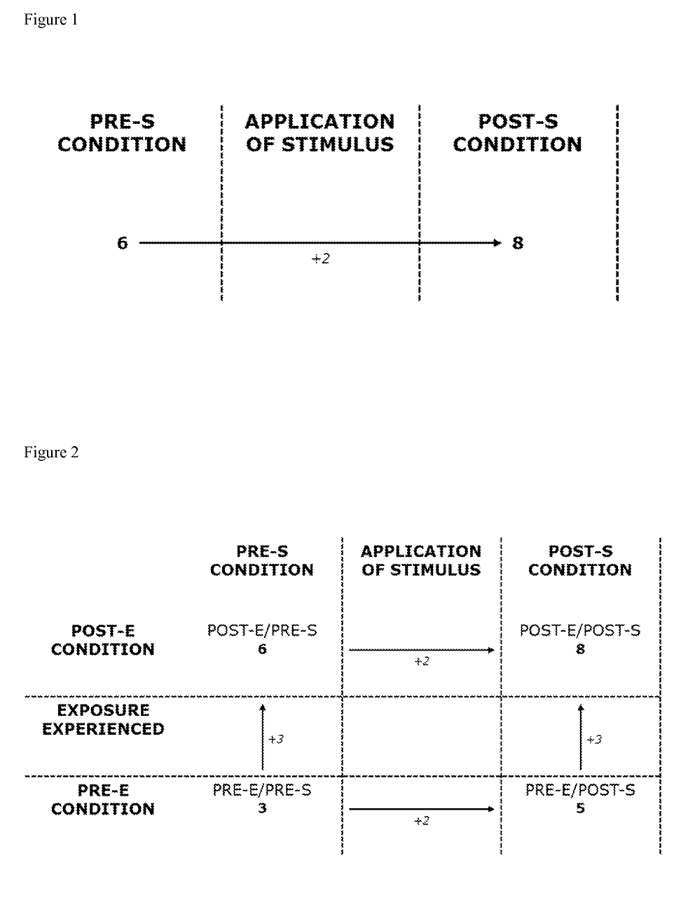

If the practitioner collects cells only from a time Post-Exposure, then the practitioner is limited to only two observational data points—Post-E/Pre-S (which we will assume that, when measured using an appropriate scale, yields a measure of 6 units), and Post-E/Post-S (which we will assume yields a measure of 8 units). The practitioner can only perform one comparison (Post-E/Post-S minus Post-E/Pre-S, or 8 minus 6, for a value of 2) (see FIG. 1). Because both observations occur Post-E, there is no information about the impact of the exposure on the stimulus-response.

If, instead, in addition to the above, the practitioner collects cells from the time period before the exposure (referred to as Pre-E), then the practitioner has data from a third condition (Pre-E/Pre-S); and, if the practitioner exposes those cells to the stimulus, the practitioner will have data from a fourth condition (Pre-E/Post-S).

The benefits of this additional data are straightforward. Assume the practitioner collects the four following measurements of the biological reaction of interest: Pre-E/Pre-S equals 3; Pre-E/Post-S equals 5; Post-E/Pre-S equals 6; and Post-E/Post-S equals 8 (see FIG. 2). In this case, the practitioner can determine that (assuming one uses an additive model) the exposure has no effect on the response to the stimulus. This is because the differences in the quantities measured do not vary between the Pre-Exposure and Post-Exposure states. That is, Pre-E/Post-S (i.e., 5) minus Pre-E/Pre-S (i.e., 3) equals 2, and Post-E/Post-S (i.e., 8) minus Post-E/Pre-S (i.e., 6) also equals 2. Therefore, while the exposure had impact on the individual, because the absolute score of the Post-E/Pre-S (i.e., 6) was larger by 3 than the Pre-E/Pre-S score (i.e., 3), and the Post-E/Post-S score (i.e., 8) was larger by 3 than the Pre-E/Post-S score (i.e., 5), the fact that these two effects were the same indicates that the exposure did not affect the degree of impact of the stimulus. The impact of the stimulus was 2 in both cases.

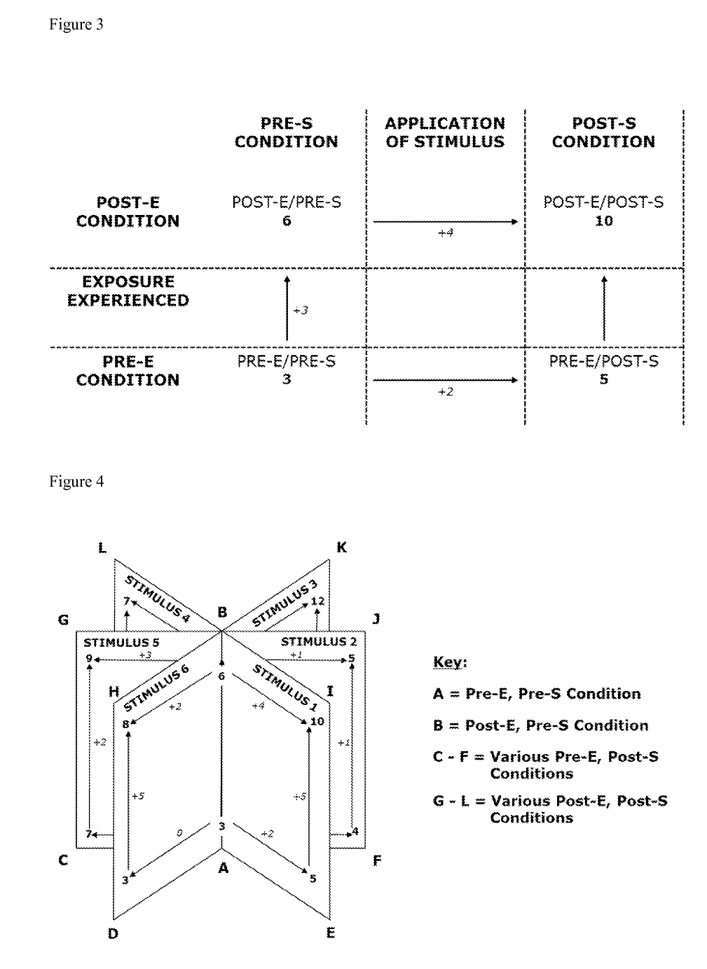

Now consider a different situation, in which the four scores are nearly the same as before, but not quite: Pre-E/Pre-S still equals 3; Pre-E/Post-S still equals 5; Post-E/Pre-S still equals 6; however, Post-E/Post-S now equals 10 instead of 8 (see FIG. 3). In this case, the practitioner can determine that the exposure does have an effect on the response to the stimulus. This is because the differences in the quantities measured vary between the Pre-Exposure and Post-Exposure states—that is, Pre-E/Post-S (i.e., 5) minus Pre-E/Pre-S (i.e., 3) still equals 2, but Post-E/Post-S (i.e., 10) minus Post-E/Pre-S (i.e. 6) equals 4. That is, the exposure to S has increased the differential by 2 (being 4 minus 2). Therefore, the exposure had impact on the response to S. The practitioner concludes that the impacts of E and S are not simply additive, as they were above, but synergistic in nature.

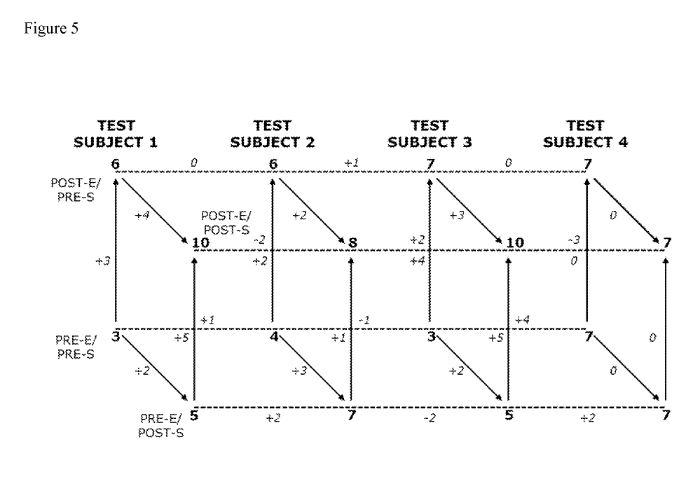

The analysis can be expanded in additional dimensions—such as analyzing multiple different stimuli on a single individual (see FIG. 4), or analyzing one stimulus against multiple test subjects who have experienced the same exposures in order to focus on differences caused by genetic diversity (see FIG. 5).

Importantly, while these examples illustrate the potential of the method for enabling insights, the method is not bound by any one mathematical approach to the analysis of the data. While the above models used the “additive” approach often found in such research into the effects of multiple factors, other models can also be applied. Indeed, the impact of each factor may not be constant across genetic differences. Each factor in the Exposome may serve as an accelerant or retardant of the impact of other factors. Further, the acceleration or retardation may be of an additive, multiplicative, exponential or other mathematical nature.

4. The Method may be Practiced in a Number of Embodiments.

While there is a single underlying structure to the method (i.e., the collection of cells from a donor at two or more points in time, and the conversion of those cells to iPSCs), the method can be practiced in multiple embodiments for different purposes. While the embodiments may vary on any of a number of dimensions, three dimensions are particularly worth noting: (a) the number of donors/test subjects; (b) the previous exposures of the initial set of cells collected from a donor; and (c) the choice of previously-stored cells versus fresh cells.

a) Number of donors/test subjects. The method may be practiced on a single individual, when there is particular interest in that individual per se, or when that individual is acting as a single point “representative” of its population. Conversely, the method may be practiced on multiple individuals when the practitioner intends to find commonalities among the individuals' reactions in order to develop normative insights for a population as a whole, or when the practitioner intends to examine the differences among the individuals and infer the impacts of genetic differences, etc. In one embodiment the number of donors is 10 or more.

b) Previous exposures of the initial set of cells collected from a donor. In some cases, such as when the test subject is about to experience a highly significant exposure (such as a high dose of radiation) and the purpose of the platform is to specifically look at the impact of that particular exposure on the test subject's subsequent response to a particular stimulus, the practitioner may be relatively indifferent to previous exposures. In these cases, the initial set of cells may be collected at any point prior to the exposure under study.

In other cases, the practitioner may not want to focus exclusively on any single source of exposure, but instead want to explore the impacts of an undetermined variety of exposures on the subsequent stimulus-response results. In this case, the practitioner may prefer a “full accounting” of all exposures that might influence the results. This may be facilitated by obtaining the first set of cells under Null-exposome Conditions. In practical terms, this may involve the use of perinatal cells as described in Patent Application Ser. No. 62/064,067, which is incorporated by reference herein.

c) Previously-stored cells versus fresh cells. One version of the platform involves collecting the initial set of cells directly from the donor, cryogenically preserving the cells (either before or after conversion to iPSCs), then waiting until a future time (after the donor has been subjected to exposures) to collect the later set of cells.

An alternative, but equally valid, version involves utilizing a previously collected and cryopreserved set of cells as the initial set, followed by utilizing either (1) another previously (but later in the donor's life) collected and cryopreserved set of cells (assuming that data is available as to the exposures the donor experienced in the interim); or (2) a set of cells collected fresh from the same donor now, or at some future date (again along with information about the exposures experienced during the interim period). For example, a practitioner could use cryogenically preserved cord blood from donors from whom the practitioner subsequently obtains fresh blood samples.

The examples below are intended to further illustrate certain aspects of the methods described herein and are not intended to limit the scope of the claims.

### EXAMPLES

### Example 1

### Analysis of the Effects of Age and Exposure to Exposome on Biological Responses to Pharmaceutical Compounds and How Genetics Can Alter These Effects

A pharmaceutical company is considering whether to seek FDA approval for several compounds that are currently on the market for use by adults for new uses in the pediatric market for similar indications. Given the length and cost of the approval process, the company desires to study in advance whether the compounds may have adverse cardiac side effects in children before deciding whether to proceed. The company is further interested in whether effects (if there are any) might be age-dependent, a result of specific genetics, and/or pertain to specific exposures to certain other pharmaceuticals.

The company seeks answers within a relatively short period of time. Therefore, the practitioner decides to build the platform based on (1) an initial collection of cells previously collected at birth and cryopreserved, and (2) current collection of cells from those same individuals. More specifically, the practitioner seeks out a private cord-blood bank that has been accepting cord blood for more than 10 years. He then obtains the list of samples collected during a particular time period that are still stored in the bank, but which have been “abandoned” by the parents. A sample is abandoned when the parents choose to stop paying to maintain the cells. The bank often does not physically remove the sample—but the parents lose any rights to demand the cells for therapy. By contacting the parents, negotiating an appropriate financial arrangement, and administering a collection process, the practitioner is able to obtain: (i) the right to use the stored cells (ii) completed questionnaires as to the child's history of exposures to medicines and vaccines since birth, and (iii) a vial of fresh blood drawn from the child at this time.

The practitioner thaws the cord blood, and extracts Endothelial Progenitor Cells (EPCs) from it. Those EPCs are converted to iPSCs using a commercially available kit (Stemgent, Cambridge, Mass.). The practitioner then extracts EPCs from the fresh peripheral blood obtained in the blood draw. These too are converted to iPSCs. These two sets of iPSCs, along with the information on exposures form the platform of the current method.

### Example 2

### Measuring the Indirect Health Impacts of a Potential Pollution

A large chemical plant is planned that will emit certain pollutants into the air. Local health officials desire to be able to discern later not just whether the pollution has affected the local population's health directly, but also whether exposure to the pollution has led to changes in the local population's reactions to other chemicals they will be exposed to, such as other sources of pollution and common pharmaceuticals.

The health officials decide to build a bio-bank with cryopreserved cells taken from a large number of the local population, along with conducting a detailed questionnaire of the individuals from whom the samples are collected as to their age and prior exposures (such as dietary habits, medicines and vaccines taken, and whether the interviewee has lived in this area throughout his/her life). They further document the environmental history of the locality in order to have an established history of the environmental exposures of the population. The information can enable the health officials later to establish the baseline exposures—i.e. the Pre-E conditions—for each individual.

Should there later be a question as to the impact of the pollution directly, or as to whether the pollution has changed the population's ability to successfully absorb subsequent stimuli (such as additional sources of pollution, or normal vaccines and pharmaceuticals), the health authorities will have a baseline of cells to create Pre-E cells (to compare to Post-E cells from the same individuals) and the documentation to establish the baseline of exposures, and hence the net exposures between the two periods of collection.

### Example 3

### Determining Potential Side Effects of Cardiac Medication on Proposed Cancer Treatment

A lung cancer patient's cancer has progressed to the state where treatments by radiation and chemotherapy are scheduled to take place. The patient's doctors plan to follow those treatments with a set of experimental medicines that have not previously been administered after such treatments. The patient's doctors want to learn, in advance of administering the medicines, whether the chest radiation treatments and chemotherapy might affect responses of the patient's heart particularly with respect to probability of arrhythmia

The practitioner does not have the option of obtaining cells that are under Null-exposome Conditions. However, by collecting the initial set of cells before the treatments, the practitioner establishes a baseline that is both Pre-Exposure (i.e. previous to the radiation and chemotherapy) and Pre-Stimulus (i.e. the patient has not begun to take the medications). Working under an appropriate protocol overseen by an internal review board (IRB), the practitioner collects a skin biopsy from under the patient's arm, as that area of the body will be later exposed to the radiation and chemotherapy. The cells from the biopsy are converted to iPSCs using a non-integrating technology from Stemgent Technologies (Stemgent, Cambridge, Mass.). The iPSCs are then cryopreserved.

The doctors record the details of the exposures to the radiation and chemotherapy (e.g. timing, duration, precise chemistry of the treatment chemicals, etc.) as those treatments are rendered, and provide these details to the practitioner.

After the radiation and chemotherapy treatments, the practitioner again takes a skin biopsy (this time from under the patient's other arm), and converts the resulting cells to IPSCs using the identical protocol as previously. The initial set of iPSCs are then thawed.

Both sets of iPSCs are then differentiated into cardiomyocytes, as per a protocol well known in the field. These cells now represent the Pre-Exposure/Pre-Stimulus (Pre-E/Pre-S) and Post-Exposure/Pre-Stimulus (Post-E/Pre-S) conditions. Both sets of cells are aliquoted into two subsets—one to be measured Pre-Stimulus, and one to be measured Post-Stimulus. The cardiac structure and behavior Pre-E/Pre-S and Post-E/Pre-S aliquots are measured through staining tests and electrophysiological tests well known to those practiced in the art. The Pre-E/Post-S and the Post-E/Post-S aliquots are treated with concentrations of the medications that correlate to the dose levels the doctors expect to administer to the patient, then their structure and behavior are measured using the same protocols as above.

The results show that while there will likely be impacts of the medicines on heart behavior (i.e., there were differences between the Pre-S and Post-S results), the differences were not exacerbated by the exposures to radiation and chemotherapy (i.e. the difference between Post-E/Post-S and Post-E/Pre-S was the same as the difference between Pre-E/Post-S and Pre-E/Pre-S). Therefore, the doctors concluded that there were unlikely to be synergistic effects.

The platforms and methods of the appended claims are not limited in scope by the specific platforms and methods described herein, which are intended as illustrations of a few aspects of the claims and any platforms and methods that are functionally equivalent are within the scope of this disclosure. Various modifications of the platforms and methods in addition to those shown and described herein are intended to fall within the scope of the appended claims. Further while only certain representative platforms and methods and aspects of these compositions and methods are specifically described, other platforms and methods and combinations of various features of the compositions and methods are intended to fall within the scope of the appended claims, even if not specifically recited. Thus, a combination of steps, elements, components, or constituents may be explicitly mentioned herein; however, all other combinations of steps, elements, components and constituents are included, even though not explicitly stated. All publications, patents, and patent applications cited herein are hereby incorporated by reference in their entireties for all purposes.

### Example 4

### Studying the Impact of Age Progression on Responses to Certain Compounds

A researcher is interested in developing a more comprehensive picture of how reactions of individuals to compounds progress with age. Until now, scientists have been limited to examining the in vivo reactions of separate cohorts of individuals who were spaced apart in age, and statistically comparing the results. This method is fraught with difficulties and limitations—such as inconsistencies in the personal histories of environmental exposures among and across the cohorts, limitations on the number of doses that can be (ethically) tested as part of the investigation, inconsistencies of doses among the test participants, etc. More importantly, such a research design provides no longitudinal data at the individual level. On the other hand, attempting to develop such progressions by following individuals across decades will—by definition—produce insights only after extended periods of time.

The researcher designs a study to compare the reactions of individuals to various compounds at two different points in that individual's age, by comparing the reactions to the same dose concentrations of the same compounds of iPSCs derived from cells taken from the same individual at two different points in time. By aggregating the results of various individuals whose starting age is different from each other, she hopes to develop insight into the progression across decades of aging in a relatively short period of time.

The researcher identifies a factory that: (1) does not utilize any known harmful chemicals (2) experiences very little turnover, and (3) is located in an isolated town in Korea that has high air and water quality. Thus, the researcher hypothesizes that the workers in this factory who continue to maintain their employment will experience similar and benign environmental conditions for an extended period of time.

With the cooperation of management and the union, the researcher solicits large numbers of volunteers from among the workers. Each volunteer agrees to participate now, and at subsequent five year periods. At each interval, the volunteer provides a blood sample, as well as medical information, such that the researcher can eliminate any volunteer from the sample if the researcher determines that prior or intervening exposures would render that volunteer's results unrepresentative.

After the initial collection, the researcher isolates and cryogenically stores the mononuclear cells (MNCs) extracted from the blood samples using techniques well known to those practiced in the art, and catalogs the information provided, eliminating from future analysis any participant whose history or medical condition warrants. The researcher then analytically divides the volunteers into narrow age cohorts (e.g. 25-28, 29-32, 33-36, etc.) between 25 and 65 years.

At the time of the second collection, the researcher again collects and processes blood and information from the sub-set of volunteers who participate in the second round. (She discards the samples of any first round volunteers who fail to participate in the second round, which may be due to discontinuation of employment, unwillingness, etc.). She further eliminates any volunteers whose intervening exposures (as reported in the second information request) would invalidate a comparison with their former self.

Thus, the researcher can now thaw the MNCs for each individual from the two time periods, and reprogram those (separately) into iPSCs, using commercially available kits, such as those provided by Reprocell-Stemgent (Lexington, Massachusetts). The stores of iPSCs thus created (or cells derived from those iPSCs) can be used to test identical dose-concentrations of compounds, and the results compared. In this way, an “individual-specific” progression can be calculated. Aggregating results for all the individuals in an age cohort can produce age range specific norms for such progression, as well as elucidating the distributions around those norms.

Because each cohort represents a different starting (and ending) age, and the five year periods between the first and second collections result in the ending age of one cohort overlapping the starting age of another cohort, the researcher is able to develop a model or models that approximate the progression across all of the age spans represented. These models may consist of a single model, or separate ones for important subsets of the volunteer population (such as gender, race, etc.).

Five years further on, the researcher undertakes a third collection. For the subset of volunteers who participated in all three rounds, the researcher is able to complete an individual progression for their fifth through tenth years, and use this data to validate and/or refine the progression model(s) developed after the second collection above.

The result of this work is a deeper understanding of how human responses to compounds change as an individual ages—including both “norms” and understanding of the distribution of such progressions.

### Example 5

### Post-Mortem Analysis of Adverse Reactions During a Clinical Trial

At the outset of a Phase III clinical trial, blood is often drawn from the prospective participants, and analyzed to determine initial medical conditions. As part of the protocol for one particular trial of a pharmaceutical compound (“Compound A”), a portion of that blood is processed to isolate the mononuclear cells (MNCs) using protocols familiar to those skilled in the art. The MNCs are then cryopreserved according to protocols that are also familiar to those skilled in the art.

During the trial, further blood samples are taken from all participants who experience any adverse drug reaction. Once again, a portion of the blood drawn is processed to isolate the MNCs, and the MNCs cryopreserved.

A number of participants experience various adverse cardiac conditions (e.g. arrhythmia, decreased heart rate, etc.) during the later stages of the trial. While comparative analysis of the incidence of such events between cases and controls is inconclusive, there appears to be some indication that the incidence of such adverse effects increased as the trial progressed.

A researcher is tasked to investigate further whether prolonged exposure to Compound A may make an individual more vulnerable to such conditions over time, through impact on the DNA in the heart cells. The researcher determines that the intravenous administration of Compound A resulted in prolonged presence of Compound A in the blood, and also has reason to believe that if Compound A produced any DNA-based cardiotoxic effect, then Compound A would affect the DNA of blood MNCs as well. Therefore, cardiotoxic testing of cells derived from prolonged-exposure blood cells might show increased sensitivity to cardiotoxic effects.

The researcher selects MNCs derived from each affected individual before the trial and MNCs derived from that same individual late in the trial and reprograms them (separately) into iPSCs using a commercial kit purchased from Reprocell-Stemgent (Lexington, Massachusetts). The researcher then differentiates the resulting iPSCs into cardiomyocytes using the protocol of U.S. Pat. No. 8,951,798.

The two sets of resulting cardiomyocytes (from each affected individual) are then challenged by several (identical between the two sets) dose-concentrations of Compound A, using assays designed to measure heart behavior, including electrophysiological measurements available from a variety of vendors (e.g. Axion Biosystems, Atlanta Georgia). By comparing the dose response curves across the two sets of experiments, the researcher is able to discern whether that particular individual would likely be more vulnerable to such cardiotoxic effects (precipitated by Compound A) after prolonged exposure to Compound A than before such exposure.

Further, aggregating such results across all of the participants who experienced such effects in vivo provides an indication as to whether the phenomenon is likely to be true across a population.

1. A method for determining the effect of age or environmental exposure on a response of two or more test samples to a stimulus, comprising, in any order:
a. applying the stimulus to a first test sample, wherein the test sample comprises one or more induced pluripotent stem cells, or one or more cells differentiated therefrom, and the induced pluripotent stem cells have been derived from cells collected from a donor,
b. detecting the response by the first test sample to the stimulus,
c. applying the stimulus to a second test sample, wherein the second test sample comprises one or more induced pluripotent stem cells, or one or more cells differentiated therefrom, and the induced pluripotent stem cells have been derived from cells subsequently collected from the same donor from whom the first test sample was collected, wherein the age of the donor or the environment to which the donor has been exposed is different between the first test sample and the second test sample,
d. detecting the response by the second test sample to the stimulus, and
e. comparing the responses of the first and second test samples to the stimulus,
wherein a difference in responses, indicates that the difference in age of the donor and/or environmental exposure on the donor affects the response of the test samples to the stimulus.

2. The method of claim 1, wherein the environment to which the donor has been exposed is a dietary, chemical, biological or environmental factor.

3. The method of claim 1, wherein the donor is human.

4. The method of claim 1, wherein the cells that are induced to become the induced pluripotent stem cells that constitute the first test sample are obtained from the donor under null-exposome conditions.

5. The method of claim 1, comprising two or more donors.

6. The method of claim 5, wherein the cells that are induced to become the induced pluripotent stem cells that constitute the first test sample from each donor are obtained under null-exposome conditions.

7. The method of claim 1, comprising 10 or more donors.

8. The method of claim 7, wherein the cells that are induced to become the induced pluripotent stem cells that constitute the first test sample from each donor are obtained under null-exposome conditions.

9. The method of claim 1, wherein one or more of the cells that are induced to become the induced pluripotent stem cells that constitute the test samples of a donor are preserved prior to applying the stimulus.

10. A platform for determining the effect of age or environmental exposure on a response of two or more test samples to a stimulus, comprising two or more sets of induced pluripotent stem cells, or cells differentiated therefrom,
wherein each set has been derived from cells collected from a single donor at a predetermined time that is different from the time of collection of cells in other sets;
and wherein the age of the donor or the environment to which the donor has been exposed is different between the first test sample and the second test sample.

11. The platform of claim 10, wherein the environment to which the donor has been exposed is a dietary, chemical, biological or environmental factor.

12. The platform of claim 10, wherein the donor is human.

13. The platform of claim 10, wherein cells that are induced to become the induced pluripotent stem cells that constitute one of the test samples are obtained from the donor under null-exposome conditions.

14. The platform of claim 10, comprising two or more donors.

15. The platform of claim 14, wherein cells that are induced to become the induced pluripotent stem cells that constitute one of the test samples from each donor is obtained under null-exposome conditions.

16. The platform of claim 10, comprising 10 or more donors.

17. The platform of claim 16, wherein cells that are induced to become the induced pluripotent stem cells that constitute one of the test samples from each donor is obtained under null-exposome conditions.

18. The platform of claim 10, wherein one or more of the cells that are induced to become the induced pluripotent stem cells that constitute test samples are preserved prior to applying the stimulus.

19. A system comprising two or more platforms of claim 10.

20. A method for determining the effect of age or environmental exposure on a response of two or more test samples to a stimulus, comprising in any order:
a. selecting a first test sample from a specified donor, wherein the test sample comprises one or more induced pluripotent stem cells, or one or more cells differentiated therefrom, and the induced pluripotent stem cells have been derived from cells collected from a donor,
b. optionally aliquoting the first test sample into two or more subsamples,
c. optionally measuring one or more parameters of the structure or biological behavior of at least one, but not all, of the first subsamples,
d. applying the stimulus to the remaining first subsample or samples,
e. measuring one or more parameters of the structure or biological behavior of those first subsamples to which the stimulus was applied,
f. selecting a subsequent sample, wherein the subsequent test sample comprises one or more induced pluripotent stem cells, or one or more cells differentiated therefrom, and the induced pluripotent stem cells have been derived from cells subsequently collected from the same donor from whom the first test sample was collected, wherein the age of the donor or the environment to which the donor has been exposed is different between the first test samples and the subsequent test samples,
g. optionally aliquoting the subsequent test sample into two or more sub-samples,
h. optionally measuring one or more parameters of the structure or biological behavior of at least one, but not all, of the subsequent sub-samples,
i. applying the stimulus to the remaining subsequent sub-sample or sub-samples,
j. measuring one or more parameters of the structure or biological behavior of those subsequent subsamples to which the stimulus was applied,
k. comparing the measurements of the subsamples from the first and second test samples to which the stimulus has been applied,
l. optionally comparing the measurements of some or all subsamples above, be they first or subsequent, and be the ones to which the stimulus has been applied or nor
wherein a difference in responses, indicates that the age of the donor or environmental exposure on the donor affects the response of the test samples to the stimulus.
